(19) United States
(12) Patent Application Publication
(10) Pub. No.: US 2025/0041317 A1
(43) Pub. Date: Feb. 6, 2025

(54) NEUROACTIVE STEROID FOR THE TREATMENT OF HUNTINGTON'S DISEASE

(71) Applicant: SAGE THERAPEUTICS, INC., Cambridge, MA (US)

(72) Inventors: Michael C. Quirk, Acton, MA (US); Aaron Michael Koenig, Wellesley, MA (US)

(21) Appl. No.: 18/719,143

(22) PCT Filed: Dec. 12, 2022
(86) PCT No.: PCT/US2022/052596

§ 371 Date: Jun. 12, 2024

Related U.S. Application Data
(60) Provisional application No. 63/289,081, filed on Dec. 13, 2021.
(60) Provisional application No. 63/321,598, filed on Mar. 18, 2022.
(60) Provisional application No. 63/404,678, filed on Sep. 8, 2022.

(51) Int. Cl. A61K 31/575; A61P 25/28

(52) U.S. Cl. CPC A61K 31/575; A61P 25/28

(57) ABSTRACT

Provided herein are methods for improving working memory and/or executive function in a subject suffering from Huntington's Disease, comprising administering to the subject SAGE-718, an investigational positive allosteric modulator of the NMDA receptor.

This application claims priority to and the benefit of U.S. Provisional Application Nos. 63/289,081, filed Dec. 13, 2021, 63/321,598, filed Mar. 18, 2022, and 63/404,678, filed Sep. 8, 2022. The disclosure of each of the foregoing applications is incorporated by reference herein in its entirety.

### BACKGROUND

Mild cognitive impairment is common in patients who have Huntington's Disease (HD) and is associated with decreased quality of life and impaired functioning. Moreover, patients with HD mild cognitive impairment (HD-MCI) are at a high risk for developing dementia.

NMDA receptors are highly expressed in the CNS and are involved in excitatory synaptic transmission. Activating these receptors contributes to synaptic plasticity in some circumstances and excitotoxicity in others. These receptors are ligand-gated ion channels that admit Ca²⁺ after binding of the neurotransmitters glutamate and glycine, and are fundamental to excitatory neurotransmission and normal CNS function. NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. Positive modulators may be useful as therapeutic agents with potential clinical uses as cognitive enhancers and in the treatment of psychiatric disorders in which glutamatergic transmission is reduced or defective (see, e.g., Horak et al., J. Neuroscience, 2004, 24(46), 10318-10325). In contrast, negative modulators may be useful as therapeutic agents with potential clinical uses in the treatment of psychiatric disorders in which glutamatergic transmission is pathologically increased (e.g., treatment resistant depression).

There remains a need for therapeutic agents that treat mild cognitive impairment associated with Huntington's Disease.

### SUMMARY

Provided herein are methods of treating Mild Cognitive Impairment (MCI) associated with Huntington's Disease comprising administering to the subject a compound having the formula

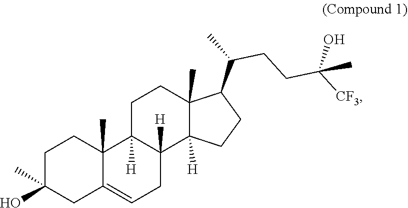

or a pharmaceutically acceptable salt thereof.

In a first aspect, provided herein is a method of treating Mild Cognitive Impairment (MCI) associated with Huntington's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In a second aspect, provided herein is a method of improving executive function in a subject having Mild Cognitive Impairment (MCI) associated with Huntington's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In a third aspect, provided herein is a method of improving working memory in a subject having Mild Cognitive Impairment (MCI) associated with Huntington's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In a fourth aspect, provided herein is a method of improving learning in a subject having Mild Cognitive Impairment (MCI) associated with Huntington's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In a fifth aspect, provided herein is a method of improving learning and working memory in a subject having MCI associated with Huntington's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In some embodiments, subject is an adult human. In some embodiments, Compound 1 or a pharmaceutically acceptable salt thereof is administered as a monotherapy. In some embodiments, Compound 1 or a pharmaceutically acceptable salt thereof is administered in combination with another therapeutic agent.

### BRIEF DESCRIPTION OF THE DRAWINGS

FIG. 1 depicts LS mean change from baseline in TWOB Test by timepoints in HD patients. Higher Scores=Better Performance on TWOB Test.

FIG. 2 depicts LS mean change from baseline in GML Test by timepoints in HD patients. Lower Scores=Better Performance on GML Test.

FIG. 3 depicts performance measured using the 2-Back Test in Patients with Huntington's Disease and Healthy Participants. Z-Transformed Change from Baseline to Day 14 (Mean±SE) is depicted.

FIG. 4 depicts performance measured using the Groton Maze Learning Test in Patients with Huntington's Disease and Healthy Participants. Z-Transformed Change from Baseline to Day 14 (Mean±SE) is depicted.

FIG. 5 depicts performance measured using the Psychomotor Vigilance Task Test in Patients with Huntington's Disease and Healthy Participants. Z-Transformed Change from Baseline to Day 14 (Mean±SE) is depicted.

### DETAILED DESCRIPTION

As generally described herein, the present invention provides compounds and compositions useful for improving cognitive function in a subject in need thereof.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with any one or more other embodiments of the invention, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (element or component) or group of integers (elements or components), but not the exclusion of any other integer (element or component) or group of integers (elements or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (element or component) or group of integers (elements or components), but not the exclusion of any other integer (elements or component) or group of integers (elements or components).

As used herein, “about” or “approximately” means within an acceptable error range for the particular value as determined by one of ordinary skill in the art, which will depend in part on how the value is measured or determined, i.e., the limitations of the measurement system.

Unless otherwise required by context, singular terms shall include pluralities and plural terms shall include the singular.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

All methods described herein can be performed in any suitable order unless otherwise indicated herein or otherwise clearly contradicted by context.

The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

As used herein, the term “unit dosage form” is defined to refer to the form in which a compound as disclosed herein (e.g., Compound 1) is administered to the subject.

As used herein, the term “daily” means a given, continuous twenty-four (24) hour period.

The terms “subject” and “patient” are used interchangeably herein and include, but are not limited to, humans and a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human (“human subject”). In certain embodiments, the human subject is an infant, child, or adolescent (“pediatric subject”). In other embodiments, the human subject is a young adult, middle-aged adult or senior adult (“adult subject”). In certain embodiments, the subject is a non-human animal (“non-human subject”).

As used herein, and unless otherwise specified, the terms “treat,” “treating” and “treatment” contemplate an action that occurs while a subject is suffering from the specified disease, disorder, or condition, which reduces the severity of the disease, disorder, or condition, or retards or slows the progression of the disease, disorder, or condition (also, “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, to delay or minimize one or more symptoms associated with the disease, disorder or condition, or to improve cognitive function. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In some embodiments, administering a compound as described herein, or a pharmaceutically acceptable salt thereof, improves cognitive function. In some embodiments, the cognitive function refers to a collection of mental tasks and functions, including but not limited to: psychomotor performance; executive performance; emotional recognition; learning (e.g, visual, spatial); memory (e.g., semantic, episodic, procedural, priming, or working); orientation; language; problem solving; visual perception, construction, and integration; planning; organizational skills; selective attention; inhibitory control; and ability to mentally manipulate information. In some embodiments, the cognitive function is one or more selected from the group consisting of psychomotor performance; executive performance; emotional recognition; learning (e.g, visual, spatial); memory (e.g., semantic, episodic, procedural, priming, or working); orientation; language; problem solving; visual perception, construction, and integration; planning; organizational skills; selective attention; inhibitory control; and ability to mentally manipulate information. In some embodiments, Compound 1 selectively targets working memory, and the improvement thereof. In some embodiments, Compound 1 selectively targets executive performance, and the improvement thereof. Measures of cognitive function include assessment tools designed to measure, for example: (a) general intelligence, (b) nonverbal intelligence, (c) achievement, (d) attention/executive functioning, (e) memory and learning, (f) visual-motor and motor functioning and (g) language.

Any change in cognitive function, for example, over time or through treatment, can be monitored by using one or more of these well-established tests at two or more time points and comparing the results. The phrase “improves cognitive function”, as referred to herein, means a positive change in the ability of the subject to perform a symbolic operation, for example, to perceive, remember, create a mental image, have clarity of thought, be aware, to reason, think or judge. The positive change can be measured using any of the aforementioned tests on two or more occasions, for example, a first occasion to measure baseline cognitive function and a second occasion to measure cognitive function following a period of time (in which treatment may have been administered). Such assessment tools are well-known in the art and include, for example, those assessment tools as described in Example 1 herein.

The term “memory” as used herein is defined as the biological processes of the brain that enable storage and recall of information.

The term “working memory” as used herein is defined as a combination of processes of the brain that provide temporary storage and manipulation of information necessary to perform complex cognitive tasks such as learning and reasoning.

The term “learning” as used herein refers to processes of the brain involved in the acquisition of skill, knowledge, and information.

The term “executive function” or “executive performance” as used interchangeably herein, refers to an umbrella term for cognitive processes that regulate, control and manage other cognitive processes, including planning, working memory, attention, problem solving, verbal reasoning, inhibition, mental flexibility, task switching, and initiation and monitoring of actions. The prefrontal areas of the frontal lobe are necessary but not sufficient for carrying out these functions.

The term “improve,” “improving,” or “improvement” or grammatical variations thereof used in relation to working memory or executive function refer to the ability to achieve a measurable increase in performance in relation to tasks used to test working memory or executive function in subjects.

### Pharmaceutical Compositions

In one aspect, the disclosure provides a pharmaceutical composition comprising a compound of the present invention (also referred to as the “active ingredient”), for example Compound 1 or a pharmaceutically acceptable salt thereof, and a pharmaceutically acceptable excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the compound or pharmaceutical composition is administered to a subject orally. In some embodiments, the compound or pharmaceutical composition is administered to a subject parenterally. In some embodiments, the compound or pharmaceutical composition is administered to a subject rectally. In some embodiments, the compound or pharmaceutical composition is administered to a subject transdermally. In some embodiments, the compound or pharmaceutical composition is administered to a subject intradermally. In some embodiments, the compound or pharmaceutical composition is administered to a subject intrathecally. In some embodiments, the compound or pharmaceutical composition is administered to a subject subcutaneously. In some embodiments, the compound or pharmaceutical composition is administered to a subject intravenously. In some embodiments, the compound or pharmaceutical composition is administered to a subject intramuscularly. In some embodiments, the compound or pharmaceutical composition is administered to a subject intranasally.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

In some embodiments, the pharmaceutical composition comprises about 0.1-about 1.0 mg of Compound 1 or a pharmaceutically acceptable salt thereof. In some embodiments, the pharmaceutical composition comprises about 0.1-about 1.0 mg of Compound 1. In some embodiments, the pharmaceutical composition comprises about 0.1-about 1.0 mg of a pharmaceutically acceptable salt of Compound 1. In some embodiments, the pharmaceutical composition comprises about 0.5 mg of Compound 1 or a pharmaceutically acceptable salt thereof. In some embodiments, the pharmaceutical composition comprises about 0.5 mg of Compound 1. In some embodiments, the pharmaceutical composition comprises about 0.5 mg of a pharmaceutically acceptable salt of Compound 1.

In some embodiments, the pharmaceutical composition comprises 0.1-1.0 mg of Compound 1 or a pharmaceutically acceptable salt thereof. In some embodiments, the pharmaceutical composition comprises 0.1-1.0 mg of Compound 1. In some embodiments, the pharmaceutical composition comprises 0.1-1.0 mg of a pharmaceutically acceptable salt of Compound 1. In some embodiments, the pharmaceutical composition comprises 0.5 mg of Compound 1 or a pharmaceutically acceptable salt thereof. In some embodiments, the pharmaceutical composition comprises 0.5 mg of Compound 1. In some embodiments, the pharmaceutical composition comprises 0.5 mg of a pharmaceutically acceptable salt of Compound 1. In some embodiments, the pharmaceutical composition comprises 1.0 mg of Compound 1 or a pharmaceutically acceptable salt thereof. In some embodiments, the pharmaceutical composition comprises 1.0 mg of Compound 1. In some embodiments, the pharmaceutical composition comprises 1.0 mg of a pharmaceutically acceptable salt of Compound 1.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

When used to prevent the onset of a CNS-disorder, the compounds provided herein will be administered to a subject at risk for developing the condition, typically on the advice and under the supervision of a physician, at the dosage levels described herein. Subjects at risk for developing a particular condition generally include those that have a family history of the condition, or those who have been identified by genetic testing or screening to be particularly susceptible to developing the condition.

### Methods of Treatment and Use

Provided herein are methods of treating Mild Cognitive Impairment (MCI) associated with Huntington's Disease comprising administering to the subject a compound having the formula

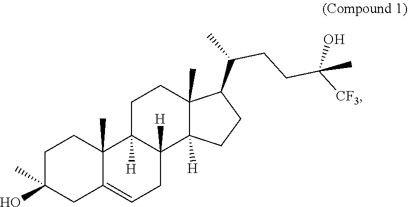

or a pharmaceutically acceptable salt thereof.

Thus, in one aspect, provided herein is a method of treating Mild Cognitive Impairment (MCI) associated with Huntington's Disease in a subject, the method comprising administering to the subject Compound 1, or a pharmaceutically acceptable salt thereof. In some embodiments, provided herein is a method of treating Mild Cognitive Impairment (MCI) associated with Huntington's Disease in a subject, the method comprising administering to the subject Compound 1. In some embodiments, provided herein is a method of treating Mild Cognitive Impairment (MCI) associated with Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of treating Mild Cognitive Impairment (MCI) resulting from Huntington's Disease in a subject, the method comprising administering to the subject Compound 1, or a pharmaceutically acceptable salt thereof. In some embodiments, provided herein is a method of treating Mild Cognitive Impairment (MCI) resulting from Huntington's Disease in a subject, the method comprising administering to the subject Compound 1. In some embodiments, provided herein is a method of treating Mild Cognitive Impairment (MCI) resulting from Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of treating Mild Cognitive Impairment (MCI) in a subject having Huntington's Disease, the method comprising administering to the subject Compound 1, or a pharmaceutically acceptable salt thereof. In some embodiments, provided herein is a method of treating Mild Cognitive Impairment (MCI) in a subject having Huntington's Disease, the method comprising administering to the subject Compound 1. In some embodiments, provided herein is a method of treating Mild Cognitive Impairment (MCI) in a subject having Huntington's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving executive function in a subject having Mild Cognitive Impairment (MCI) associated with Huntington's Disease, the method comprising administering to the subject Compound 1, or a pharmaceutically acceptable salt thereof. In some embodiments, provided herein is a method of improving executive function in a subject having Mild Cognitive Impairment (MCI) associated with Huntington's Disease in a subject, the method comprising administering to the subject Compound 1. In some embodiments, provided herein is a method of improving executive function in a subject having Mild Cognitive Impairment (MCI) associated with Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving executive function in a subject having Mild Cognitive Impairment (MCI) resulting from Huntington's Disease, the method comprising administering to the subject Compound 1, or a pharmaceutically acceptable salt thereof. In some embodiments, provided herein is a method of improving executive function in a subject having Mild Cognitive Impairment (MCI) resulting from Huntington's Disease in a subject, the method comprising administering to the subject Compound 1. In some embodiments, provided herein is a method of improving executive function in a subject having Mild Cognitive Impairment (MCI) resulting from Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving executive function in a Huntington's Disease subject having Mild Cognitive Impairment (MCI), the method comprising administering to the subject Compound 1, or a pharmaceutically acceptable salt thereof. In some embodiments, provided herein is a method of improving executive function, in a Huntington's Disease subject having Mild Cognitive Impairment (MCI), the method comprising administering to the subject Compound 1. In some embodiments, provided herein is a method of improving executive function in a Huntington's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving working memory in a subject having MCI associated with Huntington's Disease, the method comprising administering to the subject Compound 1, or a pharmaceutically acceptable salt thereof. In some embodiments, provided herein is a method of improving working memory in a subject having Mild Cognitive Impairment (MCI) associated with Huntington's Disease in a subject, the method comprising administering to the subject Compound 1. In some embodiments, provided herein is a method of improving working memory in a subject having Mild Cognitive Impairment (MCI) associated with Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving working memory in a subject having Mild Cognitive Impairment (MCI) resulting from Huntington's Disease, the method comprising administering to the subject Compound 1, or a pharmaceutically acceptable salt thereof. In some embodiments, provided herein is a method of improving working memory in a subject having Mild Cognitive Impairment (MCI) resulting from Huntington's Disease in a subject, the method comprising administering to the subject Compound 1. In some embodiments, provided herein is a method of improving working memory in a subject having Mild Cognitive Impairment (MCI) resulting from Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving working memory in a Huntington's Disease subject having Mild Cognitive Impairment (MCI), the method comprising administering to the subject Compound 1, or a pharmaceutically acceptable salt thereof. In some embodiments, provided herein is a method of improving working memory, in a Huntington's Disease subject having Mild Cognitive Impairment (MCI), the method comprising administering to the subject Compound 1. In some embodiments, provided herein is a method of improving working memory in a Huntington's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving learning in a subject having Mild Cognitive Impairment (MCI) associated with Huntington's Disease, the method comprising administering to the subject Compound 1, or a pharmaceutically acceptable salt thereof. In some embodiments, provided herein is a method of improving learning in a subject having Mild Cognitive Impairment (MCI) associated with Huntington's Disease in a subject, the method comprising administering to the subject Compound 1. In some embodiments, provided herein is a method of improving learning in a subject having Mild Cognitive Impairment (MCI) associated with Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning in a subject having Mild Cognitive Impairment (MCI) resulting from Huntington's Disease, the method comprising administering to the subject Compound 1, or a pharmaceutically acceptable salt thereof. In some embodiments, provided herein is a method of improving learning in a subject having Mild Cognitive Impairment (MCI) resulting from Huntington's Disease in a subject, the method comprising administering to the subject Compound 1. In some embodiments, provided herein is a method of improving learning in a subject having Mild Cognitive Impairment (MCI) resulting from Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning in a Huntington's Disease subject having Mild Cognitive Impairment (MCI), the method comprising administering to the subject Compound 1, or a pharmaceutically acceptable salt thereof. In some embodiments, provided herein is a method of improving learning in a Huntington's Disease subject having Mild Cognitive Impairment (MCI), the method comprising administering to the subject Compound 1. In some embodiments, provided herein is a method of improving learning in a Huntington's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving learning and working memory in a subject having Mild Cognitive Impairment (MCI) associated with Huntington's Disease, the method comprising administering to the subject Compound 1, or a pharmaceutically acceptable salt thereof. In some embodiments, provided herein is a method of improving learning and working memory in a subject having Mild Cognitive Impairment (MCI) associated with Huntington's Disease in a subject, the method comprising administering to the subject Compound 1. In some embodiments, provided herein is a method of improving learning and working memory in a subject having Mild Cognitive Impairment (MCI) associated with Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning and working memory in a subject having Mild Cognitive Impairment (MCI) resulting from Huntington's Disease, the method comprising administering to the subject Compound 1, or a pharmaceutically acceptable salt thereof. In some embodiments, provided herein is a method of improving learning and working memory in a subject having Mild Cognitive Impairment (MCI) resulting from Huntington's Disease in a subject, the method comprising administering to the subject Compound 1. In some embodiments, provided herein is a method of improving learning and working memory in a subject having Mild Cognitive Impairment (MCI) resulting from Huntington's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning and working memory in a Huntington's Disease subject having Mild Cognitive Impairment (MCI), the method comprising administering to the subject Compound 1, or a pharmaceutically acceptable salt thereof. In some embodiments, provided herein is a method of improving learning and working memory in a Huntington's Disease subject having Mild Cognitive Impairment (MCI), the method comprising administering to the subject Compound 1. In some embodiments, provided herein is a method of improving learning and working memory in a Huntington's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In some embodiments, provided herein is the use of Compound 1, or a pharmaceutically acceptable salt thereof for the manufacture of a medicament for treating Mild Cognitive Impairment (MCI) associated with Huntington's Disease in a subject. In some embodiments, provided herein is the use of Compound 1 for the manufacture of a medicament for treating Mild Cognitive Impairment (MCI) associated with Huntington's Disease in a subject. In some embodiments, provided herein is the use of a pharmaceutically acceptable salt of Compound 1 for the manufacture of a medicament for treating Mild Cognitive Impairment (MCI) associated with Huntington's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for treating Mild Cognitive Impairment (MCI) resulting from Huntington's Disease in a subject. In some embodiments, provided herein is the use of Compound 1 for the manufacture of a medicament for treating Mild Cognitive Impairment (MCI) resulting from Huntington's Disease in a subject. In some embodiments, provided herein is the use of a pharmaceutically acceptable salt of Compound 1 for the manufacture of a medicament for treating Mild Cognitive Impairment (MCI) resulting from Huntington's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for treating Mild Cognitive Impairment (MCI) in a subject having Huntington's Disease. In some embodiments, provided herein is the use of Compound 1 for the manufacture of a medicament for treating Mild Cognitive Impairment (MCI) in a subject having Huntington's Disease. In some embodiments, provided herein is the use of a pharmaceutically acceptable salt of Compound 1 for the manufacture of a medicament for treating Mild Cognitive Impairment (MCI) in a subject having Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving executive function in a subject having Mild Cognitive Impairment (MCI) associated with Huntington's Disease. In some embodiments, provided herein is the use of Compound 1 for the manufacture of a medicament for improving executive function in a subject having Mild Cognitive Impairment (MCI) associated with Huntington's Disease. In some embodiments, provided herein is the use of a pharmaceutically acceptable salt of Compound 1 for the manufacture of a medicament for improving executive function in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving executive function in a subject having Mild Cognitive Impairment (MCI) resulting from Huntington's Disease. In some embodiments, provided herein is the use of Compound 1 for the manufacture of a medicament for improving executive function in a subject having Mild Cognitive Impairment (MCI) resulting from Huntington's Disease. In some embodiments, provided herein is the use of a pharmaceutically acceptable salt of Compound 1 for the manufacture of a medicament for improving executive function in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving executive function in a Huntington's Disease subject having Mild Cognitive Impairment (MCI). In some embodiments, provided herein is the use of Compound 1 for the manufacture of a medicament for improving executive function in a Huntington's Disease subject having Mild Cognitive Impairment (MCI). In some embodiments, provided herein is the use of a pharmaceutically acceptable salt of Compound 1 for the manufacture of a medicament for improving executive function in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving working memory in a subject having Mild Cognitive Impairment (MCI) associated with Huntington's Disease. In some embodiments, provided herein is the use of Compound 1 for the manufacture of a medicament for improving working memory in a subject having Mild Cognitive Impairment (MCI) associated with Huntington's Disease. In some embodiments, provided herein is the use of a pharmaceutically acceptable salt of Compound 1 for the manufacture of a medicament for improving working memory in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving working memory in a subject having Mild Cognitive Impairment (MCI) resulting from Huntington's Disease. In some embodiments, provided herein is the use of Compound 1 for the manufacture of a medicament for method of improving working memory in a subject having Mild Cognitive Impairment (MCI) resulting from Huntington's Disease. In some embodiments, provided herein is the use of a pharmaceutically acceptable salt of Compound 1 for the manufacture of a medicament for method of improving working memory in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving working memory in a Huntington's Disease subject having Mild Cognitive Impairment (MCI). In some embodiments, provided herein is the use of Compound 1 for the manufacture of a medicament for improving working memory in a Huntington's Disease subject having Mild Cognitive Impairment (MCI). In some embodiments, provided herein is the use of a pharmaceutically acceptable salt of Compound 1 for the manufacture of a medicament for improving working memory in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving learning in a subject having Mild Cognitive Impairment (MCI) associated with Huntington's Disease. In some embodiments, provided herein is the use of Compound 1 for the manufacture of a medicament for improving learning in a subject having Mild Cognitive Impairment (MCI) associated with Huntington's Disease. In some embodiments, provided herein is the use of a pharmaceutically acceptable salt of Compound 1 for the manufacture of a medicament for improving learning in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving learning in a subject having Mild Cognitive Impairment (MCI) resulting from Huntington's Disease. In some embodiments, provided herein is the use of Compound 1 for the manufacture of a medicament for improving learning in a subject having Mild Cognitive Impairment (MCI) resulting from Huntington's Disease. In some embodiments, provided herein is the use of a pharmaceutically acceptable salt of Compound 1 for the manufacture of a medicament for improving learning in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving learning in a Huntington's Disease subject having Mild Cognitive Impairment (MCI). In some embodiments, provided herein is the use of Compound 1 for the manufacture of a medicament for improving learning in a Huntington's Disease subject having Mild Cognitive Impairment (MCI). In some embodiments, provided herein is the use of a pharmaceutically acceptable salt of Compound 1 for the manufacture of a medicament for improving learning in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving learning and working memory in a subject having Mild Cognitive Impairment (MCI) associated with Huntington's Disease. In some embodiments, provided herein is the use of Compound 1 for the manufacture of a medicament for improving learning and working memory in a subject having Mild Cognitive Impairment (MCI) associated with Huntington's Disease. In some embodiments, provided herein is the use of a pharmaceutically acceptable salt of Compound 1 for the manufacture of a medicament for improving learning and working memory in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving learning and working memory in a subject having Mild Cognitive Impairment (MCI) resulting from Huntington's Disease. In some embodiments, provided herein is the use of Compound 1 for the manufacture of a medicament for improving learning and working memory in a subject having Mild Cognitive Impairment (MCI) resulting from Huntington's Disease. In some embodiments, provided herein is the use of a pharmaceutically acceptable salt of Compound 1 for the manufacture of a medicament for improving learning and working memory in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for improving learning and working memory in a Huntington's Disease subject having Mild Cognitive Impairment (MCI). In some embodiments, provided herein is the use of Compound 1 for the manufacture of a medicament for improving learning and working memory in a Huntington's Disease subject having Mild Cognitive Impairment (MCI). In some embodiments, provided herein is the use of a pharmaceutically acceptable salt of Compound 1 for the manufacture of a medicament for improving learning and working memory in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in treating Mild Cognitive Impairment (MCI) associated with Huntington's Disease in a subject. In some embodiments, provided herein is Compound 1 for use in treating Mild Cognitive Impairment (MCI) associated with Huntington's Disease in a subject. In some embodiments, provided herein is a pharmaceutically acceptable salt of Compound 1 for use in treating Mild Cognitive Impairment (MCI) associated with Huntington's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in treating Mild Cognitive Impairment (MCI) resulting from Huntington's Disease in a subject. In some embodiments, provided herein is Compound 1 for use in treating Mild Cognitive Impairment (MCI) resulting from Huntington's Disease in a subject. In some embodiments, provided herein is a pharmaceutically acceptable salt of Compound 1 for use in treating Mild Cognitive Impairment (MCI) resulting from Huntington's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in treating a Huntington's Disease subject having Mild Cognitive Impairment (MCI). In some embodiments, provided herein is Compound 1 for use in treating a Huntington's Disease subject having Mild Cognitive Impairment (MCI). In some embodiments, provided herein is a pharmaceutically acceptable salt of Compound 1 for use in treating a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving executive function in a subject having Mild Cognitive Impairment (MCI) associated with Huntington's Disease. In some embodiments, provided herein is Compound 1 for use in improving executive function in a subject having Mild Cognitive Impairment (MCI) associated with Huntington's Disease. In some embodiments, provided herein is a pharmaceutically acceptable salt of Compound 1 for use in improving executive function in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving executive function in a subject having Mild Cognitive Impairment (MCI) resulting from Huntington's Disease. In some embodiments, provided herein is Compound 1 for use in improving executive function in a subject having Mild Cognitive Impairment (MCI) resulting from Huntington's Disease. In some embodiments, provided herein is a pharmaceutically acceptable salt of Compound 1 for use in improving executive function in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving executive function in a Huntington's Disease subject having Mild Cognitive Impairment (MCI). In some embodiments, provided herein is Compound 1 for use in improving executive function in a Huntington's Disease subject having Mild Cognitive Impairment (MCI). In some embodiments, provided herein is a pharmaceutically acceptable salt of Compound 1 for use in improving executive function in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving working memory in a subject having Mild Cognitive Impairment (MCI) associated with Huntington's Disease. In some embodiments, provided herein is Compound 1 for use in improving working memory in a subject having Mild Cognitive Impairment (MCI) associated with Huntington's Disease. In some embodiments, provided herein is a pharmaceutically acceptable salt of Compound 1 for use in improving working memory in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving working memory in a subject having Mild Cognitive Impairment (MCI) resulting from Huntington's Disease. In some embodiments, provided herein is Compound 1 for use in improving working memory in a subject having Mild Cognitive Impairment (MCI) resulting from Huntington's Disease. In some embodiments, provided herein is a pharmaceutically acceptable salt of Compound 1 for use in improving working memory in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving working memory in a Huntington's Disease subject having Mild Cognitive Impairment (MCI). In some embodiments, provided herein is Compound 1 for use in improving working memory in a Huntington's Disease subject having Mild Cognitive Impairment (MCI). In some embodiments, provided herein is a pharmaceutically acceptable salt of Compound 1 for use in improving working memory in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving learning in a subject having Mild Cognitive Impairment (MCI) associated with Huntington's Disease. In some embodiments, provided herein is Compound 1 for use in improving learning in a subject having Mild Cognitive Impairment (MCI) associated with Huntington's Disease. In some embodiments, provided herein is a pharmaceutically acceptable salt of Compound 1 for use in improving learning in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving learning in a subject having Mild Cognitive Impairment (MCI) resulting from Huntington's Disease. In some embodiments, provided herein is Compound 1 for use in improving learning in a subject having Mild Cognitive Impairment (MCI) resulting from Huntington's Disease. In some embodiments, provided herein is a pharmaceutically acceptable salt of Compound 1 for use in improving learning in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving learning in a Huntington's Disease subject having Mild Cognitive Impairment (MCI). In some embodiments, provided herein is Compound 1 for use in improving learning in a Huntington's Disease subject having Mild Cognitive Impairment (MCI). In some embodiments, provided herein is a pharmaceutically acceptable salt of Compound 1 for use in improving learning in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving learning and working memory in a subject having Mild Cognitive Impairment (MCI) associated with Huntington's Disease. In some embodiments, provided herein is Compound 1 for use in improving learning and working memory in a subject having Mild Cognitive Impairment (MCI) associated with Huntington's Disease. In some embodiments, provided herein is a pharmaceutically acceptable salt of Compound 1 for use in improving learning and working memory in a subject having Mild Cognitive Impairment (MCI) associated with Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving learning and working memory in a subject having Mild Cognitive Impairment (MCI) resulting from Huntington's Disease. In some embodiments, provided herein is Compound 1 for use in improving learning and working memory in a subject having Mild Cognitive Impairment (MCI) resulting from Huntington's Disease. In some embodiments, provided herein is a pharmaceutically acceptable salt of Compound 1 for use in improving learning and working memory in a subject having Mild Cognitive Impairment (MCI) resulting from Huntington's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In some embodiments, provided herein is Compound 1, or a pharmaceutically acceptable salt thereof for use in improving learning and working memory in a Huntington's Disease subject having Mild Cognitive Impairment (MCI). In some embodiments, provided herein is Compound 1 for use in improving learning and working memory in a Huntington's Disease subject having Mild Cognitive Impairment (MCI). In some embodiments, provided herein is a pharmaceutically acceptable salt of Compound 1 for use in improving learning and working memory in a Huntington's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

### Example 1: Effect of Compound 1 on Mild Cognitive Impairment Due to Huntington's Disease

The safety and tolerability of multiple ascending doses (MAD) of Compound 1 Oral Solution in healthy adults and subjects with early manifest Huntington's disease was assessed in a double bind, placebo-controlled Phase 1 MAD study.

Subjects were assigned sequentially to one of 3 cohorts. Part A of the study consisted of Cohorts 1 and 2, each included approximately 12 healthy subjects, with 9 subjects receiving Compound 1 Oral Solution (0.5 mg or 1.0 mg) and 3 receiving placebo. Sentinel dosing of 3 subjects was employed for Cohorts 1 and 2 in a 2:1 fashion, with 2 subjects randomized to receive Compound 1 Oral Solution and 1 subject randomized to receive placebo. The remaining 9 subjects were randomized in a 7:2 fashion to receive Compound 1 Oral Solution or placebo. Cohorts 1 and 2 were divided into 4 sub-groups of 3 subjects. Each sub-group in Part A started dosing on Day 1 over the course of 4 consecutive days, with the remaining dosing days overlapping in a staggered fashion. A maximum of 3 Part A subjects per calendar day per site will begin treatment at Day 1.

Part B of the study consisted of Cohort 3 and included approximately 10 subjects with early manifest Huntington's disease, all subjects receiving Compound 1 Oral Solution. Sentinel dosing of subjects was employed for Cohort 3.

The Screening Period began with the signing of the informed consent form (ICF) at the Screening Visit. An optional genetic sample was collected during screening. On Day −2 (Part A) or Day −1 (Part B), qualified subjects were admitted to the unit. Subjects were discharged from the unit on Day 17 after completion of all assessments, provided that the Investigator was satisfied that it is safe for the subject to be discharged. In total, the inpatient stay for Part A subjects was approximately 19 days, and approximately 18 days for Part B subjects. Subjects returned for a Follow-up Visit on Day 21 (±2 days), approximately one week after the end of treatment.

6 HD Subjects were treated with Compound 1 and completed study.

The primary objective of the study was to assess safety and tolerability of Compound 1 Oral Solution, as assessed by the incidence of adverse events (AEs)/serious adverse events (SAEs), and changes from baseline in vital signs, electrocardiograms (ECGs), clinical laboratory parameters, and Columbia-Suicide Severity Rating Scale (C-SSRS).

The secondary objective of the study was to assess the PK profile of Compound 1 following administration of multiple doses of Compound 1 Oral Solution (as assessed by area under the curve [AUC], maximum observed concentration [Cₘₐₓ], time of occurrence of Cₘₐₓ [tₘₐₓ], etc.)

Additional objectives included an assessment of changes in subject performance on cognitive tasks.

Cogstate Battery was used to assess cognitive functioning, such as attention, working memory, information processing speed, executive function, and motor skills, and consists of the Stop Signal Reaction Test (STOP), Detection Task (DET), Identification Task (IDN), One Back Learning Task (ONB), Two Back Learning Task (TWOB), and Groton Maze Learning Test (GML). Table 1.

TABLE 1

| Cogstate Test | Cognitive Domain and | | |
|---|---|---|---|
| (Test Code) | Instructions | Primary Outcome Measure | Interpretation |
| Detection Test | Psychomotor Function, | Speed of performance; mean of the | Lower score = better |
| (DET) | Has the card turned | log₁₀ transformed reaction times for | performance |
| | over? | correct responses | |
| Identification Test | Attention | Speed of performance; transformed | Lower score = better |
| (IDN) | Is the card red? | reaction times for correct responses | performance |
| One Back Test | Working Memory | Speed of performance; transformed | Lower score = better |
| (ONB) | Is the card the same as | reaction times for correct responses | performance |
| | the previous card? | | |
| Two Back Test | Working Memory | Accuracy of performance; arcsine | Higher score = better |
| (TWOB) | Is the card the same as | square root proportion correct | performance |
| | that shown two cards | | |
| | ago? | | |
| Groton Maze Learning | Executive Function | Number of errors learning the same | Lower score = better |
| Test (GML) | Find the hidden pathway. | hidden pathway across the consecutive | performance |
| | | learning trials | |
| STOP Test | Inhibition (Executive | Stop Signal Reaction Time; Difference | Lower score = better |
| (STOP) | Function) | between the mean reaction time of | performance |
| | Don't respond after a | correct responses and the stop signal | |
| | beep. | interval in which subjects inhibit their | |
| | | responses | |

The endpoints for each Cogstate Battery tests are change from baseline scores at each post-baseline assessment.

Mean change from baseline scores at all post-baseline assessments were analyzed using mixed-effect repeated measure model (MMRM); with dose, visit, interaction between dose and visit as fixed effects, baseline as a covariate and subject as random effect. Unstructured covariance structure was used to model the variance-covariance matrix of the repeated measurements. If estimation issues arose during construction of covariance structure, compound symmetry was used in place of unstructured. The least square (LS) means differences between dose groups and placebo at each post baseline timepoint were estimated along with 95% confidence intervals.

The magnitude of the differences between Compound 1 dose groups and placebo, the effect size, was also assessed using Cohen's d (Cohen, 1988), using the following formula:

Cohen's d score=(Mean of experimental group−Mean of control group)/Pooled standard deviation (SD) for entire sample.

The effect size: The magnitude of the differences between treatment groups and placebo: <0.2 considered as trivial, 0.2-0.5 considered as small, 0.5-0.8 considered as moderate, 0.8-1.1 considered as large, >1.1 considered as very large.

### Results

### Safety Summary:

No deaths were reported
No serious AEs were reported
No TEAEs leading to study withdrawal or treatment discontinuation were reported
No TEAEs were reported in Part B in subjects with HD
There were no signals for increased suicidal ideation or suicidal behavior compared to baseline, as measured by C-SSRS.

### Changes in HD Subject Performance on Cognitive Tasks

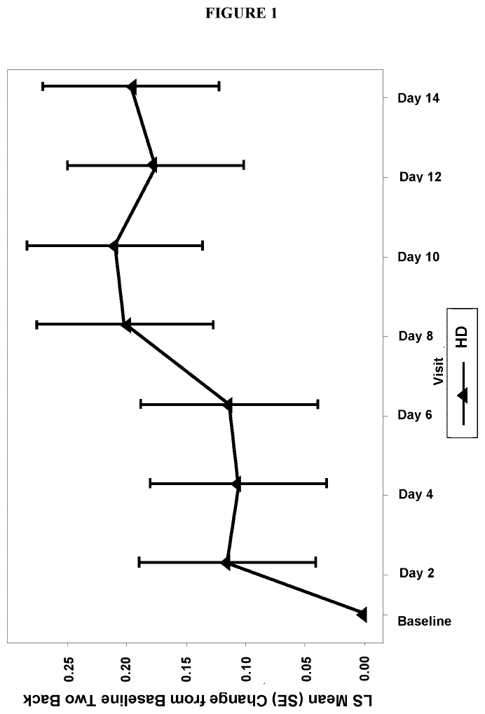

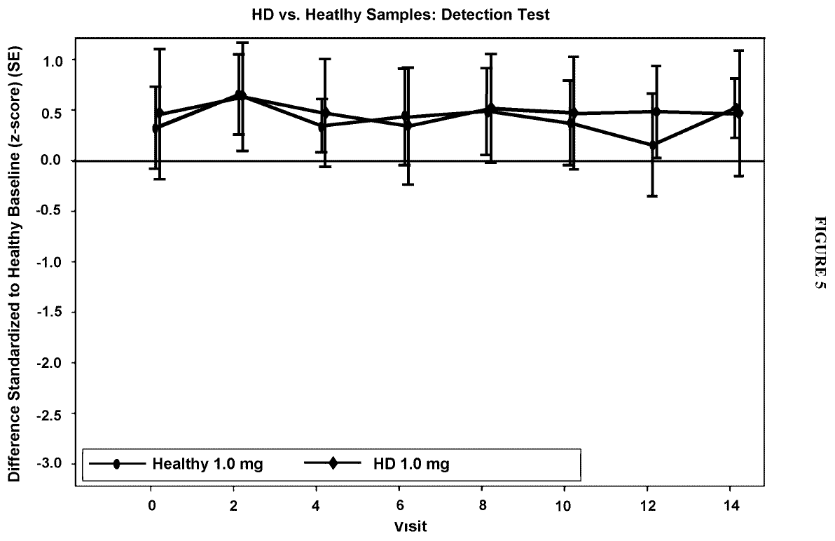

Patients with HD who received Compound 1 (1 mg) exhibited improvements from baseline on tests of executive functioning (see below and FIGS. 1-4). No changes from baseline were observed on assessment of psychomotor speed (see FIG. 5).

### A. Two Back Test

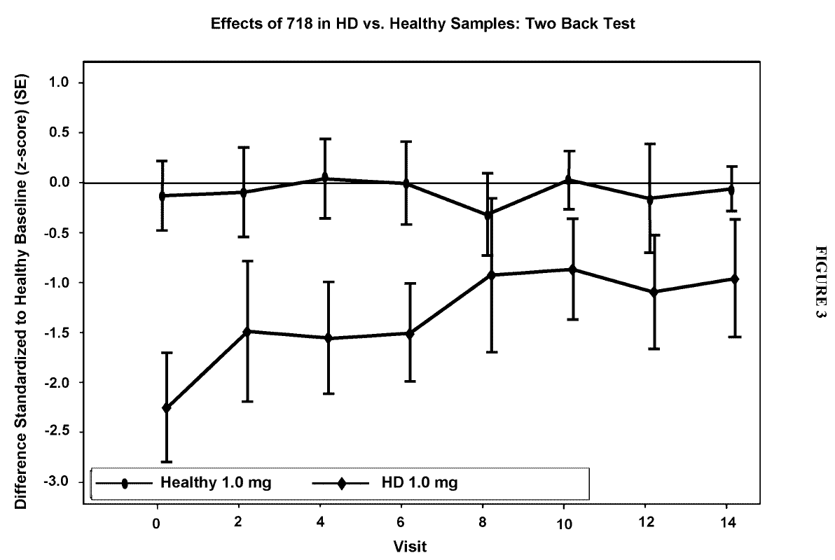

HD Subjects (Part B) demonstrated improvement in performance on the two-back test after being treated with Compound 1 (1 mg) at all timepoints after baseline and met threshold (p<0.05) for significant improvement compared to baseline on Days 8-14. The LS mean change from baseline in Two-Back (TWOB) Test by timepoints are shown in FIGS. 1 and 3.

### B. Groton Maze Learning (GML) Test:

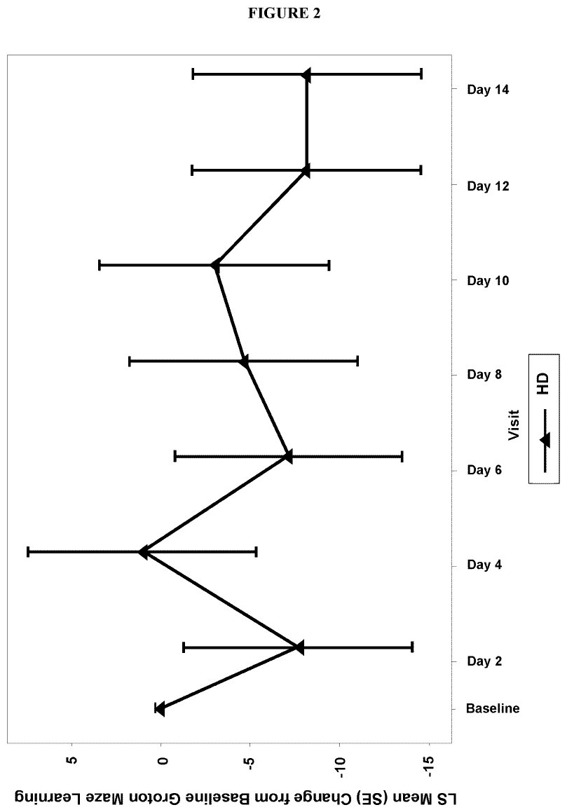

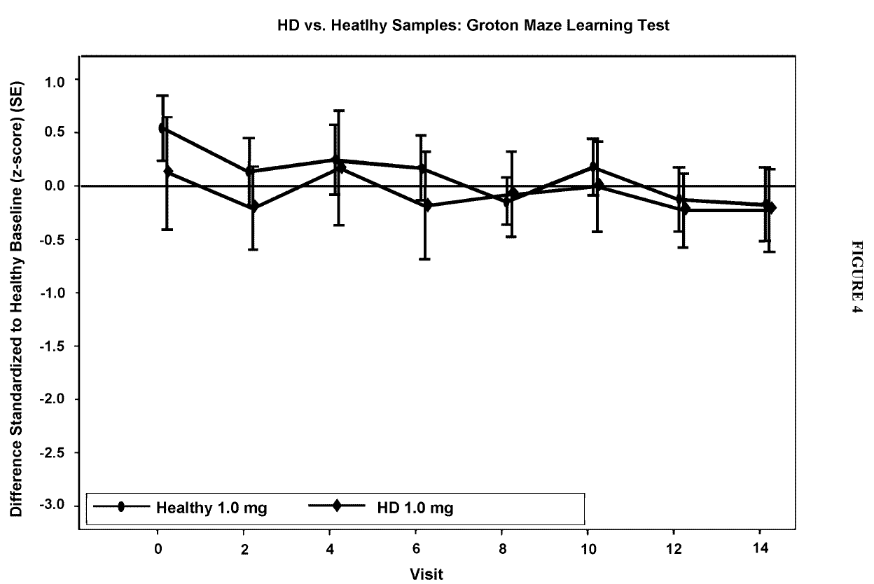

HD Subjects (Part B) demonstrated improvement in the GML test after being treated with Compound 1 (1 mg) at all timepoints except for day 4. Mean change from baseline in GML Test by timepoints are shown in FIGS. 2 and 4.

### C. Huntington's Disease Subjects Baseline Cognitive Test:

HD Subjects demonstrate worse baseline cognitive performance compared with healthy subjects, meeting the threshold for statistical significance (p<0.05) for the One-Back, Two-Back, and Stop Signal tests. Table 2 shows the results of baseline cognitive tests comparison b/w HD Subjects and healthy subjects.

TABLE 2

| | Healthy Subjects | HD subjects | | | | |
|---|---|---|---|---|---|---|
| | Baseline | Baseline | Difference | | p- | Effect |
| Test (unit) | (mean, SD) | (mean, SD | in Means | 95% CI | value | Size |
| Detection Test | 2.54 (0.093) | 2.58 (0.146) | 0.043 | (−0.055, 0.141) | 0.373 | 0.413 |
| Score (DET) | | | | | | |
| (log10 msec) | | | | | | |
| Groton Maze | 57.54 (23.0) | 60.5 (29.71) | 2.958 | (−19.797, 25.714) | 0.792 | 0.122 |
| Learning Test | | | | | | |
| Score (GML) | | | | | | |
| (count) | | | | | | |
| Identification | 2.70 (0.076) | 2.72 (0.121) | 0.016 | (−0.065, 0.096) | 0.691 | 0.183 |
| Test Score (IDN) | | | | | | |
| (log10 msec) | | | | | | |
| One-Back Test | 2.83 (0.075) | 2.92 (0.159) | 0.094 | (0.005, 0.183) | 0.040 | 0.984 |
| Score (ONB) | | | | | | |
| (log10 msec) | | | | | | |
| Two-Back Test | 1.27 (0.151) | 0.93 (0.203) | −0.341 | (−0.492, −0.190) | <0.001 | −2.106 |
| Score (TWOB) | | | | | | |
| (arcsin | | | | | | |
| sqrt(correct/total) | | | | | | |
| STOP Signal | 211.5(45.60) | 356.2 (202.87) | 144.7 | (53.941, 235.5) | 0.003 | 1.499 |
| Test (msec) | | | | | | |

### EQUIVALENTS

Those skilled in the art will recognize or will be able to ascertain using no more than routine experimentation, numerous equivalents to the compounds, compositions, and methods of use thereof described herein. Such equivalents are considered to be within the scope of the invention.

### NUMBERED EMBODIMENTS

Certain embodiments of the disclosure are set forth in the following numbered paragraphs:

1. A method of treating Mild Cognitive Impairment (MCI) associated with Huntington's Disease in a subject, comprising administering to the subject a compound having the formula

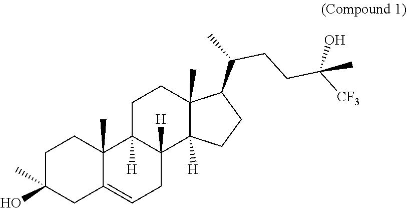

or a pharmaceutically acceptable salt thereof.

2. The method of embodiment 1, wherein Compound 1 or a pharmaceutically acceptable salt thereof is administered to the subject daily.

3. The method of embodiment 1 or 2, wherein Compound 1 or a pharmaceutically acceptable salt thereof is administered to the subject at a dose of about 3 mg/day.

4. The method of embodiment 3, wherein about 3 mg daily of Compound 1 is administered to the subject.

5. The method of any one of embodiments 1-4, wherein Compound 1 or a pharmaceutically acceptable salt thereof is administered orally.

6. A method of improving executive function in a subject having Mild Cognitive Impairment (MCI) associated with Huntington's Disease, the method comprising administering to the subject a compound having the formula

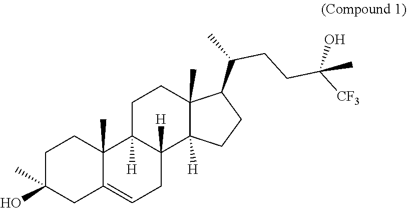

or a pharmaceutically acceptable salt thereof.

7. The method of embodiment 6, wherein Compound 1 or a pharmaceutically acceptable salt thereof is administered to the subject daily.

8. The method of embodiment 6 or 7, wherein Compound 1 or a pharmaceutically acceptable salt thereof is administered to the subject at a dose of about 3 mg/day.

9. The method of embodiment 8, wherein about 3 mg daily of Compound 1 is administered to the subject.

10. The method of any one of embodiments 6-9, wherein Compound 1 or a pharmaceutically acceptable salt thereof is administered orally.

11. A method of improving working memory in a subject having Mild Cognitive Impairment (MCI) associated with Huntington's Disease, the method comprising administering to the subject a compound having the formula

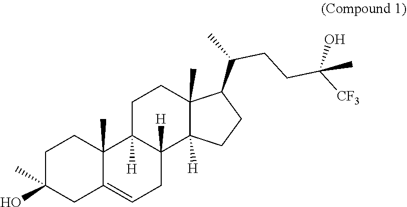

or a pharmaceutically acceptable salt thereof.

12. The method of embodiment 11, wherein Compound 1 or a pharmaceutically acceptable salt thereof is administered to the subject daily.

13. The method of embodiment 11 or 12, wherein Compound 1 or a pharmaceutically acceptable salt thereof is administered to the subject at a dose of about 3 mg/day.

14. The method of embodiment 13, wherein about 3 mg daily of Compound 1 is administered to the subject.

15. The method of any one of embodiments 11-14, wherein Compound 1 or a pharmaceutically acceptable salt thereof is administered orally.

16. A method of improving learning in a subject having Mild Cognitive Impairment (MCI) associated with Huntington's Disease, the method comprising administering to the subject a compound having the formula

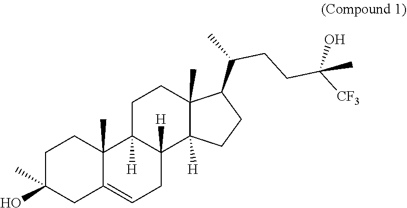

or a pharmaceutically acceptable salt thereof.

17. The method of embodiment 16, wherein Compound 1 or a pharmaceutically acceptable salt thereof is administered to the subject daily.

18. The method of embodiment 16 or 17, wherein Compound 1 or a pharmaceutically acceptable salt thereof is administered to the subject at a dose of about 3 mg/day.

19. The method of embodiment 18, wherein about 3 mg daily of Compound 1 is administered to the subject.

20. The method of any one of embodiments 16-19, wherein Compound 1 or a pharmaceutically acceptable salt thereof is administered orally.

21. A method of improving learning and working memory in a subject having Mild Cognitive Impairment (MCI) associated with Huntington's Disease, the method comprising administering to the subject a compound having the formula

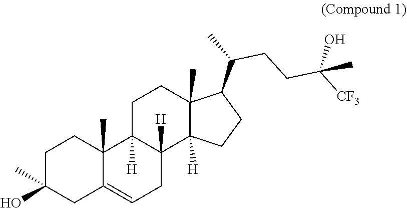

or a pharmaceutically acceptable salt thereof.

22. The method of embodiment 21, wherein Compound 1 or a pharmaceutically acceptable salt thereof is administered to the subject daily.

23. The method of embodiment 21 or 22, wherein Compound 1 or a pharmaceutically acceptable salt thereof is administered to the subject at a dose of about 3 mg/day.

24. The method of embodiment 23, wherein about 3 mg daily of Compound 1 is administered to the subject.

25. The method of any one of embodiments 21-24, wherein Compound 1 or a pharmaceutically acceptable salt thereof is administered orally.

26. The method of any one of embodiments 1-25, wherein the subject is an adult.

27. A compound having the formula

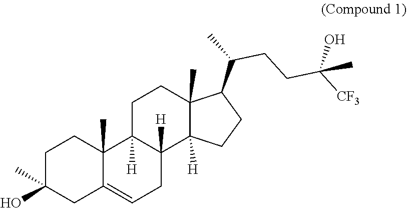

or a pharmaceutically acceptable salt thereof, for use in treating Mild Cognitive Impairment (MCI) associated with Huntington's Disease in a subject.

28. The compound for use according to embodiment 27, wherein Compound 1 or a pharmaceutically acceptable salt thereof is administered to the subject daily.

29. The compound for use according to embodiment 27 or 28, wherein Compound 1 or a pharmaceutically acceptable salt thereof is administered to the subject at a dose of about 3 mg/day.

30. The compound for use according to embodiment 29, wherein about 3 mg daily of Compound 1 is administered to the subject.

31. The compound for use according to any one of embodiments 27-30, wherein Compound 1 or a pharmaceutically acceptable salt thereof is administered orally.

32. A compound having the formula

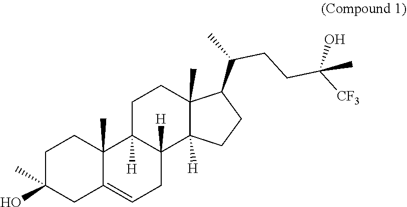

or a pharmaceutically acceptable salt thereof, for use in improving executive function in a subject having Mild Cognitive Impairment (MCI) associated with Huntington's Disease.

33. The compound for use according to embodiment 32, wherein Compound 1 or a pharmaceutically acceptable salt thereof is administered to the subject daily.

34. The compound for use according to embodiment 32 or 33, wherein Compound 1 or a pharmaceutically acceptable salt thereof is administered to the subject at a dose of about 3 mg/day.

35. The compound for use according to embodiment 34, wherein about 3 mg daily of Compound 1 is administered to the subject.

36. The compound for use according to any one of embodiments 32-35, wherein Compound 1 or a pharmaceutically acceptable salt thereof is administered orally.

37. A compound having the formula

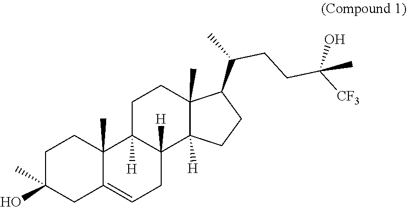

or a pharmaceutically acceptable salt thereof, for use in improving working memory in a subject having Mild Cognitive Impairment (MCI) associated with Huntington's Disease.

38. The compound for use according to embodiment 37, wherein Compound 1 or a pharmaceutically acceptable salt thereof is administered to the subject daily.

39. The compound for use according to embodiment 37 or 38, wherein Compound 1 or a pharmaceutically acceptable salt thereof is administered to the subject at a dose of about 3 mg/day.

40. The compound for use according to embodiment 39, wherein about 3 mg daily of Compound 1 is administered to the subject.

41. The compound for use according to any one of embodiments 37-40, wherein Compound 1 or a pharmaceutically acceptable salt thereof is administered orally.

42. A compound having the formula

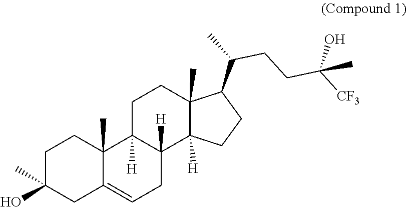

or a pharmaceutically acceptable salt thereof, for use in improving learning in a subject having Mild Cognitive Impairment (MCI) associated with Huntington's Disease.

43. The compound for use according to embodiment 42, wherein Compound 1 or a pharmaceutically acceptable salt thereof is administered to the subject daily.

44. The compound for use according to embodiment 42 or 43, wherein Compound 1 or a pharmaceutically acceptable salt thereof is administered to the subject at a dose of about 3 mg/day.

45. The compound for use according to embodiment 44, wherein about 3 mg daily of Compound 1 is administered to the subject.

46. The compound for use according to any one of embodiments 42-45, wherein Compound 1 or a pharmaceutically acceptable salt thereof is administered orally.

47. A compound having the formula

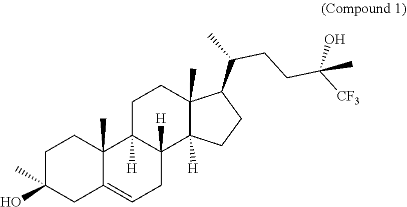

or a pharmaceutically acceptable salt thereof, for use in improving learning and working memory in a subject having Mild Cognitive Impairment (MCI) associated with Huntington's Disease.

48. The compound for use according to embodiment 47, wherein Compound 1 or a pharmaceutically acceptable salt thereof is administered to the subject daily.

49. The compound for use according to embodiment 47 or 48, wherein Compound 1 or a pharmaceutically acceptable salt thereof is administered to the subject at a dose of about 3 mg/day.

50. The compound for use according to embodiment 49, wherein about 3 mg daily of Compound 1 is administered to the subject.

51. The compound for use according to any one of embodiments 47-50, wherein Compound 1 or a pharmaceutically acceptable salt thereof is administered orally.

52. The compound for use according to any one of embodiments 27-51, wherein the subject is an adult.

53. Use of a compound having the formula

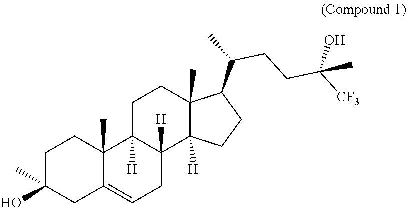

or a pharmaceutically acceptable salt thereof, for the preparation of a medicament for treating Mild Cognitive Impairment (MCI) associated with Huntington's Disease in a subject.

54. The use according to embodiment 53, wherein Compound 1 or a pharmaceutically acceptable salt thereof is administered to the subject daily.

55. The use according to embodiment 53 or 54, wherein Compound 1 or a pharmaceutically acceptable salt thereof is administered to the subject at a dose of about 3 mg/day.

56. The use according to embodiment 55, wherein about 3 mg daily of Compound 1 is administered to the subject.

57. The use according to any one of embodiments 53-56, wherein Compound 1 or a pharmaceutically acceptable salt thereof is administered orally.

58. Use of a compound having the formula

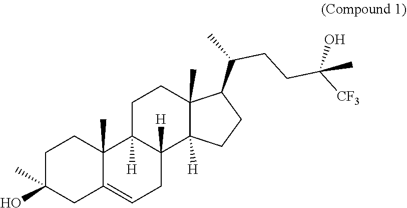

or a pharmaceutically acceptable salt thereof, for the preparation of a medicament for improving executive function in a subject having Mild Cognitive Impairment (MCI) associated with Huntington's Disease.

59. The use according to embodiment 58, wherein Compound 1 or a pharmaceutically acceptable salt thereof is administered to the subject daily.

60. The use according to embodiment 58 or 59, wherein Compound 1 or a pharmaceutically acceptable salt thereof is administered to the subject at a dose of about 3 mg/day.

61. The use according to embodiment 60, wherein about 3 mg daily of Compound 1 is administered to the subject.

62. The use according to any one of embodiments 58-61, wherein Compound 1 or a pharmaceutically acceptable salt thereof is administered orally.

63. Use of a compound having the formula

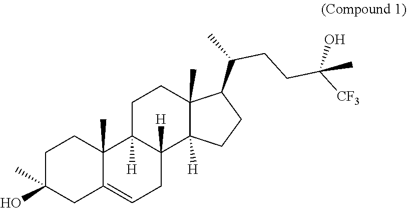

or a pharmaceutically acceptable salt thereof, for the preparation of a medicament for improving working memory in a subject having Mild Cognitive Impairment (MCI) associated with Huntington's Disease.

64. The use according to embodiment 63, wherein Compound 1 or a pharmaceutically acceptable salt thereof is administered to the subject daily.

65. The use according to embodiment 63 or 64, wherein Compound 1 or a pharmaceutically acceptable salt thereof is administered to the subject at a dose of about 3 mg/day.

66. The use according to embodiment 65, wherein about 3 mg daily of Compound 1 is administered to the subject.

67. The use according to any one of embodiments 63-66, wherein Compound 1 or a pharmaceutically acceptable salt thereof is administered orally.

68. Use of a compound having the formula

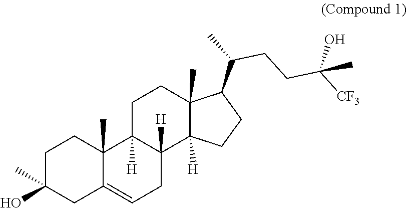

or a pharmaceutically acceptable salt thereof, for the preparation of a medicament for improving learning in a subject having Mild Cognitive Impairment (MCI) associated with Huntington's Disease.

69. The use according to embodiment 68, wherein Compound 1 or a pharmaceutically acceptable salt thereof is administered to the subject daily.

70. The use according to embodiment 68 or 69, wherein Compound 1 or a pharmaceutically acceptable salt thereof is administered to the subject at a dose of about 3 mg/day.

71. The use according to embodiment 70, wherein about 3 mg daily of Compound 1 is administered to the subject.

72. The use according to any one of embodiments 68-71, wherein Compound 1 or a pharmaceutically acceptable salt thereof is administered orally.

73. Use of a compound having the formula

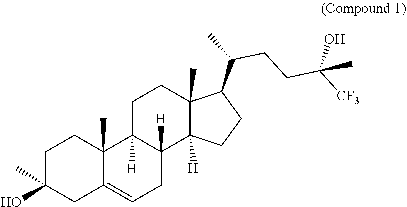

or a pharmaceutically acceptable salt thereof, for the preparation of a medicament for improving learning and working memory in a subject having Mild Cognitive Impairment (MCI) associated with Huntington's Disease.

74. The use according to embodiment 73, wherein Compound 1 or a pharmaceutically acceptable salt thereof is administered to the subject daily.

75. The use according to embodiment 73 or 74, wherein Compound 1 or a pharmaceutically acceptable salt thereof is administered to the subject at a dose of about 3 mg/day.

76. The use according to embodiment 75, wherein about 3 mg daily of Compound 1 is administered to the subject.

77. The use according to any one of embodiments 73-76, wherein Compound 1 or a pharmaceutically acceptable salt thereof is administered orally.

78. The use according to any one of embodiments 53-77, wherein the subject is an adult.

1. A method of treating Mild Cognitive Impairment (MCI) associated with Huntington's Disease in a subject, comprising administering to the subject a compound having the formula 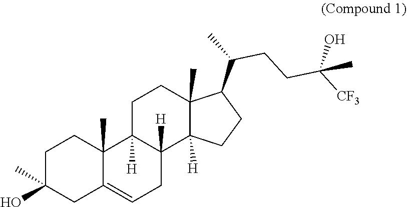
or a pharmaceutically acceptable salt thereof.

2. A method of improving executive function in a subject having Mild Cognitive Impairment (MCI) associated with Huntington's Disease, the method comprising administering to the subject a compound having the formula 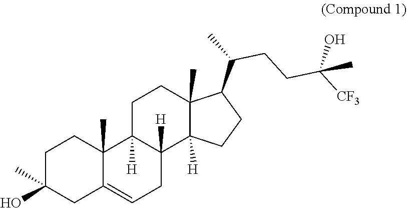
or a pharmaceutically acceptable salt thereof.

3. A method of improving working memory in a subject having Mild Cognitive Impairment (MCI) associated with Huntington's Disease, the method comprising administering to the subject a compound having the formula 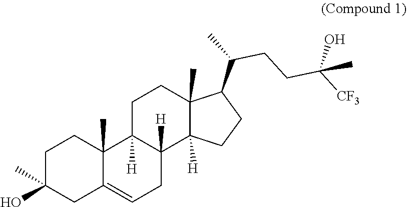
or a pharmaceutically acceptable salt thereof.

4. A method of improving learning in a subject having Mild Cognitive Impairment (MCI) associated with Huntington's Disease, the method comprising administering to the subject a compound having the formula 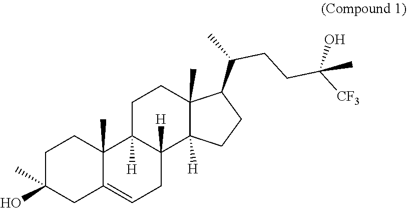
or a pharmaceutically acceptable salt thereof.

5. A method of improving learning and working memory in a subject having Mild Cognitive Impairment (MCI) associated with Huntington's Disease, the method comprising administering to the subject a compound having the formula 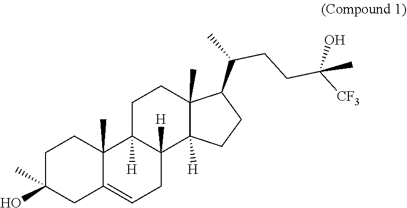
or a pharmaceutically acceptable salt thereof.

6. The method of any one of claims 1-5, wherein Compound 1 or a pharmaceutically acceptable salt thereof is administered to the subject daily.

7. The method of any one of claims 1-6, wherein Compound 1 or a pharmaceutically acceptable salt thereof is administered to the subject at a dose of about 3 mg/day.

8. The method of claim 7, wherein about 3 mg daily of Compound 1 is administered to the subject.

9. The method of any one of claims 1-8, wherein Compound 1 or a pharmaceutically acceptable salt thereof is administered orally.

10. A compound having the formula 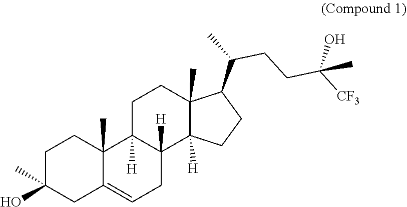
or a pharmaceutically acceptable salt thereof, for use in treating Mild Cognitive Impairment (MCI) associated with Huntington's Disease in a subject.

11. A compound having the formula 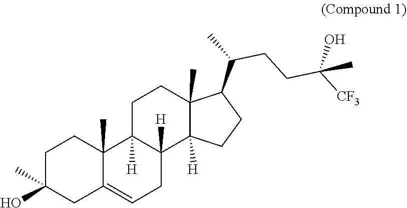
or a pharmaceutically acceptable salt thereof, for use in improving executive function in a subject having Mild Cognitive Impairment (MCI) associated with Huntington's Disease.

12. A compound having the formula 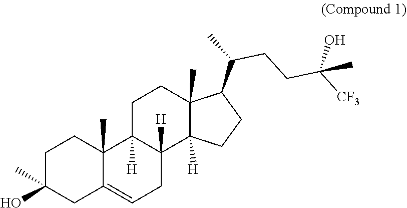
or a pharmaceutically acceptable salt thereof, for use in improving working memory in a subject having Mild Cognitive Impairment (MCI) associated with Huntington's Disease.

13. A compound having the formula 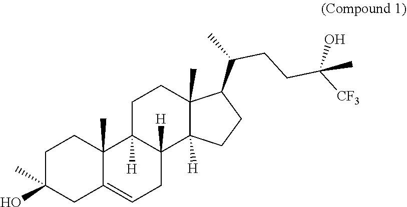
or a pharmaceutically acceptable salt thereof, for use in improving learning in a subject having Mild Cognitive Impairment (MCI) associated with Huntington's Disease.

14. A compound having the formula 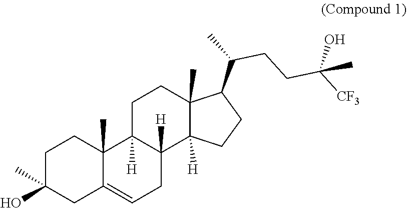
or a pharmaceutically acceptable salt thereof, for use in improving learning and working memory in a subject having Mild Cognitive Impairment (MCI) associated with Huntington's Disease.

15. The compound for use according to any one of claims 10-14, wherein Compound 1 or a pharmaceutically acceptable salt thereof is administered to the subject daily.

16. The compound for use according to any one of claims 10-15, wherein Compound 1 or a pharmaceutically acceptable salt thereof is administered to the subject at a dose of about 3 mg/day.

17. The compound for use according to claim 16, wherein about 3 mg daily of Compound 1 is administered to the subject.

18. The compound for use according to any one of claims 10-17, wherein Compound 1 or a pharmaceutically acceptable salt thereof is administered orally.

19. Use of a compound having the formula 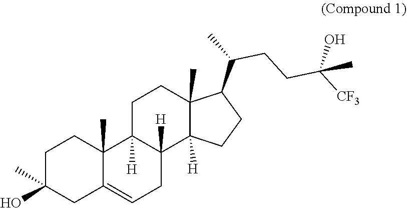
or a pharmaceutically acceptable salt thereof, for the preparation of a medicament for treating Mild Cognitive Impairment (MCI) associated with Huntington's Disease in a subject.

20. Use of a compound having the formula 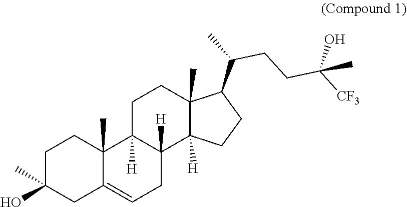
or a pharmaceutically acceptable salt thereof, for the preparation of a medicament for improving executive function in a subject having Mild Cognitive Impairment (MCI) associated with Huntington's Disease.

21. Use of a compound having the formula 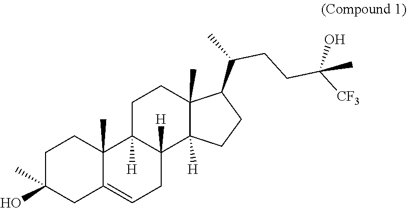
or a pharmaceutically acceptable salt thereof, for the preparation of a medicament for improving working memory in a subject having Mild Cognitive Impairment (MCI) associated with Huntington's Disease.

22. Use of a compound having the formula 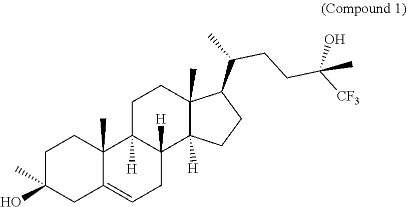
or a pharmaceutically acceptable salt thereof, for the preparation of a medicament for improving learning in a subject having Mild Cognitive Impairment (MCI) associated with Huntington's Disease.

23. Use of a compound having the formula 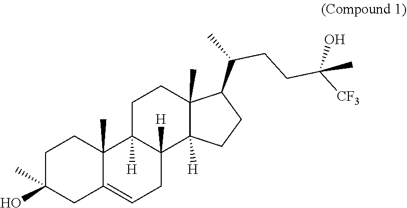
or a pharmaceutically acceptable salt thereof, for the preparation of a medicament for improving learning and working memory in a subject having Mild Cognitive Impairment (MCI) associated with Huntington's Disease.

24. The use according to any one of claims 19-23, wherein Compound 1 or a pharmaceutically acceptable salt thereof is administered to the subject daily.

25. The use according to any one of claims 19-24, wherein Compound 1 or a pharmaceutically acceptable salt thereof is administered to the subject at a dose of about 3 mg/day.

26. The use according to claim 25, wherein about 3 mg daily of Compound 1 is administered to the subject.

27. The use according to any one of claims 19-26, wherein Compound 1 or a pharmaceutically acceptable salt thereof is administered orally.
